(19) United States
(12) Patent Application Publication
(10) Pub. No.: US 2025/0082510 A1
(43) Pub. Date: Mar. 13, 2025

(54) WOUND DRESSING

(71) Applicant: Essity Hygiene and Health Aktiebolag, Goteborg (SE)

(72) Inventor: Kolja Szymanski, Hamburg (DE)

(21) Appl. No.: 18/729,049

(22) PCT Filed: Feb. 22, 2022
(86) PCT No.: PCT/EP2022/054464

§ 371 Date: Jul. 15, 2024

(51) Int. Cl. A61F 13/0206; A61F 13/02

(52) U.S. Cl. CPC A61F 13/0206; A61F 13/0276

(57) ABSTRACT

Disclosed is a skin dressing for topical delivery of an active agent, such as nitric oxide, including a covering layer, an absorbent layer, a first compartment including a first reactant, wherein the first reactant is a precursor of the active agent, a second compartment including a second reactant, and a proximal layer permeable to the active agent; wherein the first compartment has a central opening.

### CROSS REFERENCE TO RELATED APPLICATIONS

This application is a National Stage Application of PCT/EP2022/054464, filed Feb. 22, 2022, which is incorporated by reference in its entirety herein.

### TECHNICAL FIELD

The present disclosure relates to a dressing for topical delivery of an active agent, such as nitric oxide (NO), for use in the treatment of skin, in particular wounds, such as chronic wounds. The disclosure further relates to the use of said wound care product for treatment of acute and chronic wounds. The disclosure finally relates to a method of producing the wound care product.

### BACKGROUND

Wound healing is a global medical concern with several challenges, including the increasing incidence of obesity and type II diabetes and an ageing population leading to an increase in chronic wounds. Chronic wounds include, but are not limited to, diabetic ulcers, vascular (venous and arterial) ulcers and pressure ulcers. Overcoming the factors that contribute to the delayed wound healing in chronic wounds is one of the main challenges in the treatment of chronic wounds.

Wound healing is a complex process involving phases of coagulation, inflammation, removal of damaged matrix components, followed by cellular proliferation and migration, angiogenesis, matrix synthesis and deposition, re-epithelization, and remodeling. Possible barriers to wound healing which disturb the usual wound healing cascade range from hypoxia to infection.

Several wound dressings are known in the art which are designed to deliver an active agent to the wound to support or restore proper wound healing. Active agents which have been proposed to aid in the wound healing include silver, polyhexamethylene biguanide (PHMB, polyhexanide) and nitric oxide (NO). These agents have antimicrobial activity and assist the wound healing.

For example, EP 2 533 773 B1 describes a NO delivery device including a first compartment containing an amount of a reducing agent and an agent capable of forming a transient complex with nitric oxide and a second compartment containing an amount of a nitric oxide precursor that forms NO when contacted with and reduced by the reducing agent. A barrier separates the contents of the two compartments until the wound dressing is applied to the wound. EP 2 142 181 B1 describes a negative pressure wound dressing including activation fluid in a breakable pouch on top of an absorbent that includes an NO donor. The activation fluid is distributed within the absorbent after breaking the pouch by applying sub atmospheric pressure.

The treatment of chronic wounds, such as chronic wounds like diabetic foot ulcers (DFU), is often further complicated by high amounts of exudate which is exuded by the wound due to prolonged inflammatory healing phases. If the exudate is not managed adequately, leakage leads to damage of the wound margins and surrounding skin and thus to a higher pain, extension of the wound and prolonged healing. Retention of wound fluids within the wound dressing whilst ensuring a moist wound environment is a key factor of modern wound dressings. EP 3 338 813 B1 describes a multilayered wound product including an upper liquid-absorbing layer and an active agent releasing layer which have co-aligned pores through which wound exudate can reach the liquid-absorbing layer. The active agent is released from the area between the pores to the wound site.

If the wound dressings which deliver active agents, such as NO, to the wound site, shall be stored for longer periods of time, measures need to be taken to ensure that the agent, such as NO is not released prematurely during storage. Many materials contain enough moisture themselves to initiate a premature NO release from an NO donor. Thus, shelf life of a NO releasing wound dressing may be limited.

There remains a need in the art to provide absorbent wound dressings that are able to deliver an active agent evenly to a wound site and have a long shelf-life.

### SUMMARY

This problem is solved by a skin dressing according to the present disclosure as described in the appended claims. These wound dressings encapsulate a precursor of the active agent securely so that no leakage occurs before the date of use. Additionally, the wound dressings are able to distribute the active agent particularly evenly over the wound site covered by the dressing with the help of a first compartment that has a central opening. At the same time, an efficient transport of exudate away from the wound may be facilitated through pores in the dressing layers and through the central opening of the first compartment as described in more detail below.

In a first aspect, the disclosure relates to a skin dressing for topical delivery of an active agent, such as nitric oxide, including a covering layer; an absorbent layer; a first compartment including a first reactant, wherein the first reactant is a precursor of the active agent; a second compartment including a second reactant; and a proximal layer permeable to the active agent; wherein the first compartment has a central opening.

The first reactant and the second reactant included in the first compartment and second compartment, respectively, can react with each other in the absence or presence of additional reaction partners and thereby form the active agent. To avoid that this reaction is already happening during production, transport or storage of the skin dressing, the first and second compartment are separated from each other. It will be described elsewhere herein that the separation is achieved by the walls of the second compartment which act as a barrier for the first and second reactant because they are impermeable to both. Unless the second compartment is actively opened, the first reactant and the second reactant will not be in direct contact with each other.

An “active agent” in the sense of the present disclosure is an agent that has a therapeutic, cosmetic and/or preventative effect on the body of the wearer of the dressing, e.g. on the skin or wound located proximally of the dressing.

The skin dressing can, thus, in one embodiment be for use in a method for treatment or prevention of a disease or condition. The disease or condition can be a wound, such as a skin wound, in particular a chronic wound, haemorrhoids, a condition in need of improved blood circulation under the skin or wound, a condition in need of a better supply of nutrients, infection of the skin or wound, in particular bacterial infection of the skin or wound, and combinations thereof. The active agent can, for example, have a wound healing promoting effect. The skin dressing can, accordingly, be a wound dressing. In alternative embodiments, the skin dressing can be for use in cosmetic applications, such as wrinkle treatment, improving skin appearance, or the like.

The active agent will usually be an agent that reacts chemically with one or more compounds in the body of the wearer of the dressing, in particular in the skin or a wound. It is, for example known in the art that the active agent nitric oxide (NO) can have a therapeutic effect, e.g. an antimicrobial effect and wound-healing promoting effect, as well as a cosmetic effect. NO acts inter alia vasodilatory and, thus, can improve blood circulation in a wound or the skin.

The active agent may be provided in gaseous form or dissolved in a liquid. The active agent can be a gas that can preferably be selected from the group consisting of oxygen, ozone, carbon dioxide, nitric oxide or combinations thereof. In a preferred embodiment, the gas is nitric oxide (NO, nitrogen monoxide). As described above, nitric oxide is known to have a healing effect in wounds. In addition to the aforementioned gaseous active agents, the dressing can include additional active agents to provide further advantageous effects. These will be described elsewhere herein.

Since the direction of the diffusion of a gas or liquid is controlled by the principle of minimum diffusion resistance, the diffusion takes place in the direction of the least barriers. The active agent, hence, escapes substantially only through the proximal layer in the direction of the skin or wound. Thus, the active agent released inside the dressing will be directed towards the skin of the wearer of the dressing through the active agent-permeable proximal layer. The release of the active agent is effectively targeted to the skin or wound target site. The dressing can have the ability to release 0.1 nmol active agent or more to the wound or skin when applied to the wound and/or skin. It will be understood that the active agent will only be released after activation of the dressing by breaking the second compartment and releasing the second reactant onto the first compartment.

The active agent can be a type of agent that is endogenously present in the human body, such as nitric oxide.

The first compartment included in the skin dressing includes a first reactant which can, together with the second reactant included in the second compartment react to form the active agent. The first reactant is, in other words, a precursor of the active agent. The term “precursor” as used herein describes a compound (first reactant) that participates in the chemical reaction that produces another compound (active agent). Accordingly, also the second reactant can be regarded to be a precursor of the active agent. In addition to the first reactant and the second reactant, additional compounds may be involved in the reaction to form the active agent.

As mentioned before, nitric oxide is a particularly preferred active agent in the context of the present disclosure. First and second reactants which can react to form nitric oxide are well known in the art and described e.g. in EP 2 533 773 B1 which is incorporated herein by reference in its entirety.

The first reactant can, for example be a nitric oxide (NO) donor, such as sodium nitrite. In a preferred embodiment a first reactant is an NO donor and the second reactant is a reducing agent or a proton donor, wherein a reducing agent is preferred.

The NO donor can be selected from the group consisting of inorganic nitrite salts, alkyl nitrites such as isopentyl nitrite, diazeniumdiolated organic compounds, trans[RuCl([15]aneN₄)NO]²⁺, Nitrosyl ligands, 6-nitrobenzo[a]pyrol, S-nitroso-glutathione, S-nitroso-thiols, S-nitroso-N-acetyl-D-penicillamine (SNAP), L-arginine, L-citrulline, nitroglycerin (GTN), isosorbide 5-mononitrate (ISMN), isosorbide dinitrate (ISDN), pentaerythritol tetranitrate (PETN), erythrityl tetranitrate (ETN), amino acid derivatives such as N-hydroxy-L-arginine (NOHA), N.sup.6-(liminoethyl) lysine) (L-NIL), L-N.sup.5-(1-iminoethyi) ornithine (LN-NIO), N.sup.a-methyl-L-arginine (L-NMMA), S-nitroso glutathione (SNOG), S,S-dinitrosodithiol (SSDD), 2-[(pyridin-3-ylcarbonyl)amino]ethyl nitrate (nicorandil), sodium nitroprusside (SNP), S-nitroso-N-acetylpenicillamine (SNAP), [8-[2-hydroxy-3-(propan-2-ylamino) propoxy]-3,4-dihydro-2H-chromen-3-yl]nitrate (Nipradilol), 3-morpholino-sydnonimine (SIN-1), molsidomine, DEA-NONOate (2-(N,N-diethylamino)diazenolate-2-oxide), spermine NONOate (N-[4-[1-(3-aminopropyl)-2-hydroxy-2-nitrosohydrazino]butyl-1,3-propanediamine), 3-(5′-hydroxymethyl-2′furyl)-1-benzyl indazole (YC-1), nitroaniline derivatives, 2-methyl-2-nitrosopropane, imidazoyl derivatives, nitrate ester, hydroxyl nitrosamine, hydroxylamine and hydroxyl urea, and combinations thereof.

For example, the NO donor can be an inorganic nitrite salt. In a preferred embodiment, the inorganic nitrite salt is selected from the group consisting of LiNO₂, NaNO₂, KNO₂, RbNO₂, CsNO₂, FrNO₂, Be(NO₂)₂, Mg(NO₂)₂, Ca(NO₂)₂, Sr(NO₂)₂, Ba(NO₂)₂, and Ra(NO₂)₂ and combinations thereof. In particular embodiments, the inorganic nitrite salt is sodium nitrite (NaNO₂).

The NO donor, such as the inorganic nitrite salt, is present in the dressing in a therapeutically, cosmetically or preventatively effective amount. It can, for example, be present in an amount of from 10 μM to 1500 μM, from 25 μM to 1000 μM, from 50 μM to 500 μM, from 80 μM to 250 μM, and from 100 μM to 150 μM. In other words, the NO donor can be present in the dressing, specifically in the first compartment, in an amount of 10 μM or more, 25 μM or more, 50 μM or more, 75 μM or more, or 100 μM or more. The NO donor can be present in the dressing in an amount of 1500 μM or less, 1000 μM or less, 500 μM or less, 250 μM or less, 180 μM or less, or 150 μM or less.

The first compartment—in which the first reactant is included—has a central opening. In other words, the first compartment has the shape of a frame, such as a picture frame (picture frame shape). The opening ensures that, for example, wound exudate can be quickly transported from the proximal end of the dressing into the absorbent layer without being obstructed by the first compartment. The transport can be facilitated by channels which are described elsewhere herein. These channels can lead through the central opening within the first compartment.

At the same time, the first compartment is designed in a manner that it extends parallel to the proximal layer over a substantial portion of the dressing. The size and reach of the first compartment ensure that the active agent will be released over the entire surface of the proximal layer and not only locally on one end of the dressing. A “compartment” as used herein is a defined and distinct volume of gaseous, fluid and/or solid material. The first compartment, in particular the shape of the first compartment, is suitable to ensure an even distribution of the active agent throughout the skin dressing. Therefore, if the dressing is virtually divided into four quadrants that are adjacent to each other in a plane that is parallel to the proximal layer, the first compartment extends through all four virtual quadrants. In other words, the maximal width of the first compartment parallel to the proximal layer can be 50% or more, 60% or more, 70% or more, 80% or more, 90% or more of the width of the dressing in the same direction, specifically from 50% to 95%, from 60% to 95%, from 70% to 95%, or from 80% to 95%.

The opening in the first compartment is located in a middle region of the dressing. The opening is parallel to the proximal layer, i.e. it extends from the proximal to the distal side of the first compartment. The opening can, for example have a central axis in common with or parallel to a central axis (middle axis) of the skin dressing, wherein it is preferred that the opening and the skin dressing have the central axis in common. The central axis is orthogonal to the proximal layer of the dressing, i.e. extends in proximal to distal direction.

The shape of the first compartment is suitable to distribute the reactants and agents included therein and/or generated therein evenly through the dressing. At the same time, the opening in the middle of the first compartment leaves sufficient space for channels for the transport of wound exudates.

The first compartment can have an annular (circular) shape, i.e. the shape of a ring. Alternatively, its shape can be rectangular, with sharp or rounded corners; or oval. The first compartment can also have an irregular shape, a zigzag shape or the shape of the number 8. Each of the aforementioned shapes describes the respective shape parallel to the proximal layer. In proximal to distal direction, the first compartment has preferably the shape of a flat sheet or layer.

The opening can have the same shape than the compartment itself. For example, the first compartment and the opening can have a circular shape. Alternatively, the first compartment and the opening can have a rectangular shape, with sharp or rounded corners.

The outer diameter of the first compartment can be from 50% or more of the outer diameter of the skin dressing, in particular 60% or more, 70% or more, 80% or more. The “diameters” mentioned here are measured in parallel to the proximal layer. In other words, the outer diameter of the first compartment can be from 50% to 98% of the outer diameter of the skin dressing, for example from 60% to 98%, from 70% to 98%, from 80% to 98%.

The two diameters which are compared to derive a relationship (in %) between the respective diameters are measured along a common axis. For example, the outer diameter of the first compartment as measured along a line extending from a lateral to a medial side of the skin dressing can be from 50% or more of the outer diameter of the skin dressing as measured along the same line.

The diameter of the opening (i.e. the inner diameter of the first compartment) can be from 10% or more of the outer diameter of the skin dressing, in particular from 15% or more, from 20% or more, from 25% or more, from 30% or more, from 35% or more, from 40% or more, from 45% or more, from 50% or more. In other words, the diameter of the opening can be from 10% to 80% of the outer diameter of the skin dressing, from 15% to 80%, from 20% to 80%, from 25% to 80%, from 30% to 80%, from 35% to 80%, from 40% to 80%, from 45% to 80%. The diameter of the opening can, thus, be up to 80% of the outer diameter of the skin dressing, such as up to 75%, up to 70%, up to 65%, up to 60%, up to 55%, up to 50%, up to 45%, up to 40%, up to 35% of the skin dressing.

The diameter of the opening (i.e. the inner diameter of the first compartment) can be from 15% or more of the outer diameter of the first compartment, in particular from 20% or more, from 25% or more, from 30% or more, from 35% or more, from 40% or more, from 45% or more, from 50% or more. In other words, the diameter of the opening can be from 15% to 90% of the outer diameter of the first compartment, in particular from 20% to 90%, from 25% to 90%, from 30% to 90%, from 35% to 90%, from 40% to 90%, from 45% to 90%. The diameter of the opening can, thus, be up to 90% of the outer diameter of the first compartment, such as up to 85%, up to 80%, up to 75%, up to 70%, up to 65%, up to 60%, up to 55%, up to 50%, up to 45% of the skin dressing.

The opening can have a size of from 25% or more of the size of the dressing, such as from 30% or more, from 35% or more, from 40% or more, from 45% or more, from 50% or more, from 60% or more, from 70% or more. The term “size” refers to the area (e.g. as measured in cm²) of the opening or dressing measured in a plane that is parallel to the proximal layer. In other words, the opening can have a size of from 25% to 85% of the size of the dressing (in a plane that is parallel to the proximal layer), of from 30% to 85%, from 35% to 85%, from 40% to 85%, from 45% to 85%, from 50% to 85%, from 55% to 85%, from 60% to 85%, from 65% to 85%.

It will be understood that the size of the first compartment in absolute values depends on the dressing size that is needed. The larger dressing, the larger the outer diameter of the first compartment can be. The first compartment can, for example have an outer diameter (as measured parallel to the proximal layer, same applies to the following paragraphs) of from 4 cm to 10 cm, from 5 cm to 9 cm, or preferably from 6 cm to 8 cm. In other words, the first compartment can have an outer diameter of 4 cm or more, 5 cm or more, or preferably 6 cm or more.

The inner diameter of the first compartment, i.e. a diameter of the opening, can be from 3 cm to 9 cm, from 4 cm to 8 cm, or preferably from 5 cm to 7 cm. In other words, the inner diameter of the first compartment can be 3 cm or more, 4 cm or more, or preferably 5 cm or more. The inner diameter can be e.g. 0.4 cm or more shorter than the outer diameter of the first compartment, or 0.5 cm or more, 0.6 cm or more, 0.7 cm or more, or preferably 0.8 cm or more.

Such a first compartment is e.g. suitable to distribute the active agent throughout a dressing that has a diameter of from 5 cm to 15 cm, from 7 cm to 13 cm, or preferably from 9 cm to 11 cm. Accordingly, the outer diameter of the first compartment may be e.g. 0.5 cm or more shorter than the outer diameter of the dressing, or 2 cm or more, such as from 0.5 cm to 4 cm, from 1 cm to 3 cm.

The first compartment can include an absorbent material or consist substantially of an absorbent material, the first reactant, solvents and/or further reactants. The absorbent material will ensure that the first compartment-which includes the first reactant—can also quickly take up the second reactant after the second compartment is broken and that the second reactant is quickly distributed throughout the first compartment. Suitable materials can, for example, be selected from the group consisting of non-woven materials, foams or the like.

The absorbent material has a high wicking rate and high absorptive capacity. To support wicking of fluid by the material of the first compartment, the material may have pores, perforations, channels or the like.

The absorbent material can, for example, have a water absorption capacity of 200 g/m² or more, 300 g/m² or more, 400 g/m² or more, 500 g/m² or more, 600 g/m² or more, 700 g/m² or more or 800 g/m² or more. In other words, the absorbent material can have water absorption capacity of from 200 g/m² to 1600 g/m², from 300 g/m² to 1500 g/m², from 400 g/m² to 1400 g/m², from 500 g/m² to 1300 g/m², from 600 g/m² to 1200 g/m², from 700 g/m² to 1100 g/m², or from 800 g/m² to 1000 g/m². In preferred embodiments, the absorbent material is a non-woven material.

As mentioned before, the first compartment can include solvents and/or further reactants, in particular solvents and/or reactants participating in the generation of the active agent. The compartment can, for example, include the non-woven material and an aqueous solution soaked into the non-woven and including a first reactant, such as an NO-donor.

The reactant may be distributed evenly throughout the first compartment or it may be localized at a particular desired location, e.g. a location adjacent to the second compartment. This may, for example be achieved by dripping a small volume of a solution including the first reactant onto the respective location of the first compartment before assembly of the dressing.

The non-woven material can be e.g. selected from the group consisting of viscose, Polyethylene terephthalate (PET) and combinations thereof. Fibre bonding in the non-woven material may be by methods known to persons skilled in the art. For example, the non-woven may have spunlace bound fibres.

The first reactant, for example an NO donor, such as sodium nitrite, can be impregnated on the material forming the first compartment, e.g. the non-woven.

The dressing is flexible and therefore able to adapt to the skin surface to ensure good contact. The term “flexible” means that the dressing is capable of being flexed or bent without breaking. It is able to adapt to the curvature of a body part and/or a wound underneath the dressing.

The second compartment-which includes the second reactant—can be a container. The second compartment can also be termed “capsule”. It has a wall and an inner cavity. While the cavity provides room to hold the second reactant, the walls of the container make sure that the second reactant does not leak to the outside of the container unless the container walls are purposely broken. For this purpose, the walls of the container surround the cavity completely. The second reactant is, thus, preferably included in an inner cavity of the container.

The container is a breakable receptacle. Its walls can, thus, be broken by external force, e.g. by pressing and/or twisting the container within the dressing. Breaking of the container wall will create one or more holes in the walls. Thereby, the second reactant and, potentially, further compounds included in the second compartment are released into the surrounding dressing, in particular onto the first compartment. Thus, the first reactant, e.g. the NO donor, is activated, the reaction between the first and the second reactant is initiated and the release of the active agent, e.g. the NO, is initiated. In preferred embodiments, the container is, hence, adapted to be breakable by external force, specifically by force exerted by a human hand.

To facilitate deposition of the second reactant within the container during manufacture of the dressing, the container forming the second compartment may include more than one wall part. For example, the container can include a first wall part and a second wall part. The different wall parts, e.g. the first and the second wall part, are together enclosing the inner cavity. The person skilled in the art will be able to devise suitable wall parts that are able to encircle an inner cavity. One or both wall parts may have an essentially flat border region encircling the entire wall part and a depression (pit) in the middle of the wall part. After closure of the second compartment, the depression(s) will (together) form the cavity holding the second reactant. In particular embodiments, one wall part has an essentially flat border region encircling the entire wall part and a depression in its middle and another wall part is a substantially flat, shee-like structure in toto. While the former wall part can be termed “burst pouch reservoir”, the latter can be termed “burst pouch lid”.

The wall parts can be connected by sealing, preferably heat sealing. Alternative methods are known in the art. For example, the wall parts may alternatively be glued together with an adhesive or clamped together. It is also possible to screw, fold, and/or punch the first and the second wall part together. The connection between the wall parts is substantially liquid-impermeable and preferably also gas-impermeable. The connection between the two wall parts will be established in a border region surrounding the middle of each part.

The solution or composition including the second reactant can be filled into the depression(s) before the first wall part and the second wall part are sealed together. It has been shown to be particularly advantageous if at least 80%, 85% or preferably 90% of the volume of the depression(s) are filled with the solution or composition before sealing the two wall parts together. In other words, from 80% to 100%, from 85% to 100%, or from 90% to 100% of the volume of the depression(s) can be filled with the solution or composition before sealing the two wall parts together.

The second compartment can have an elongated form. Thereby, it is possible to orient the second compartment alongside of the first compartment within the dressing. The two compartments can, thus, have maximal overlap. This means, firstly, that the compartments are advantageously directly adjacent to each other. Secondly, the surface area of the second compartment that is directly adjacent to the first compartment (e.g. touches the first compartment) can be chosen to be as large as possible given the second compartment's size and shape. In other words, one of the sides of the compartment with the larger or the largest surface area is the side that is directly adjacent to the first compartment. An optimal proximity between first and the second compartment can also be achieved by a kidney-shape that the second compartment may have and/or the shape of a partial circle or a partial ellipse. The edges of the second compartment may be rounded to ensure that the compartment does not cut into any other pieces of the dressing.

To make breaking of the second compartment easier, the compartment may have a predetermined breaking point or area. This is a point or area in which the cohesion of the compartment is reduced in comparison with the remainder of the dressing. Accordingly, the compartment will predominantly break at this point or area. Thereby, the release of the solution or composition including the second reactant onto the first compartment can be targeted. The predetermined breaking point or area may, for example, be the area in which the first and the second wall part are connected with each other. The seal between the two wall parts may be designed to be weaker than the remainder of the compartment wall. The structural robustness in the breaking point or area may be >1.5 N/cm.

The wall parts may include or consist of various materials. The wall parts of the container are preferably liquid impermeable. Additionally, the wall parts of the container may also be impermeable or substantially impermeable to gas (i.e. gas-tight). Thereby, premature evaporation or leakage of the first reactant from the compartment is prevented and the shelf-life of the dressing improved. Moreover, gases contained in the environment such as oxygen are prevented from entering the first compartment and react with the reactants included therein. For example, the wall parts may include or consist of a material selected from the group consisting of glass, aluminium, aluminium alloys, aluminium composites, plastics, and combinations thereof. In some embodiments, the first wall part and/or the second wall part include or consist of an aluminium or aluminium alloy.

The wall parts may include or consist of multiple layers. At least one of these layers can include or consist of one of the aforementioned materials, such as glass, aluminium, aluminium alloys, plastics and combinations thereof. The additional layers can be chosen to improve the stability, liquid- and/or gas-impermeability and/or the ability to be laminated of the wall parts. For example, the wall parts may consist of 1 or more, 2 or more, 3 or more layers. In other words, the wall parts can consist of 1 to 5, or 2 to 4 layers. Additional layers may, e.g., include or consist of polyethylene, polyethylenterephthalate, polyamide or combinations thereof.

The thickness of the wall parts will be chosen to ensure gas- and liquid-permeability and breakability by the force of a hand (e.g. with one or more fingers) of a user. The wall parts can, for example, have a thickness of 20 μm or more, 30 μm or more, 40 μm or more, or 50 μm or more. In other words, the wall parts can have a thickness of from 20 μm to 300 μm, from 30 μm to 280 μm, from 40 μm to 250 μm, or preferably from 50 μm to 200 μm. These wall parts can e.g. be wall parts including an aluminium or alluminium alloy layer.

The size of the second compartment will be chosen to be sufficient to accommodate the required volume of second reactant. The second compartment can, for example have a length of from 1 cm to 6 cm, from 2 to 5 cm, or preferably from 3 to 4 cm. This compartment can, further have a width of from 0.5 cm to 3 cm, from 1 cm to 2.5 cm, or preferably from 1.5 cm to 2 cm. The aforementioned length and width are measured in a plane that is parallel to the proximal layer in the dressing. The height of the second compartment (as measured perpendicular to the proximal layer) can be from 0.3 cm to 2 cm, from 0.4 cm to 1 cm, or preferably from 0.5 cm to 0.8 cm.

The second reactant is an agent suitable to react with the first reactant and, thereby, produce the active agent.

The second reactant can, for example, be a reducing agent, in particular an agent able to react with the first reactant and reduce the first reactant. The reducing agent can be selected from the group consisting of ascorbic acid, cysteine, glutathione, penicillamine, N-acetylcysteine, iodine, hydroquinone, mercaptosuccinic acid, thiosalicylic acid, methylthiosalicylic acid, dithiothreitol, dithioerythritol, 2-mercaptoethanol, FeSO₄, and FeCl₂, and combinations thereof. The second reactant may be a reducing nitrosyl complex forming agent, such as Cu, Cu(I), V(III), Mo(VI), Fe(II), I—, Ti(III), Co(II), Mn(II), and Cr(III) and their salts. Preferably, the second reactant is a salt of Fe(II), such as FeSO₄ preferably Fe(II)SO₄. In particular embodiments, the second compartment, thus, includes liquid ferrous sulfate solution.

In a preferred embodiment the NO as an active agent is produced by the reaction of a NO donor with a reducing agent capable of forming a transient nitrosyl complex. NO precursors include nitrite, nitric acid, and nitrate as described elsewhere herein.

Examples of chemical reactions for producing NO include:

3NaNO₂+3FeSO₄+3H₂O→Fe₂(SO₄)3+3NaOH+FeOH₃+3NO; (1)

2NaNO₂+2FeSO₄[Fe(II)SO₄]+2H₂SO₄→Fe₂(SO₄)₃[Fe(III)₂(SO₄)₃]+2NaHSO₄+2H₂O+2NO; (2)

2NaNO₂+2KI+3H₂SO₄→12+4KHSO₄+2NO; (3)

8HNO₃+3Cu→3Cu(NO₃)₂+4H₂O+2NO; (4)

2NaNO₂+2H+→2HNO₂→N₂O₃+H₂O, N₂O₃+AA→NO+ascorbyl radical; (5)

NO₂⁻+H⁺→HNO₂,NO₂⁻+HNO₂→N₂O₃+OH⁻,N₂O₃→NO₂+NO; (6)
wherein AA=ascorbic acid.

Examples of reducing nitrosyl complex forming agents include, but are not limited to, Cu, Cu (I), V (III), Mo (VI), Fe (II), I—, Ti (III), Co (II), Mn (II), and Cr (III) and their salts. Some reducing nitrosyl complex forming agents, such as Fe (II), change colour when they form nitrosyl complexes, allowing the activation of NO production and depletion to be monitored.

The first and second reactant, for example the NO donor and reducing agent, may be in the form a solid, aqueous solution or gel. The first reactant is preferably solid. It can, for example, be added to the first compartment in solution (e.g. solved in water) and then left to dry on the first compartment. The second reactant is preferably present in a solution.

In alternative embodiments, a NO releasing polymer, for example as described in WO 2008/116497, WO 2008/116925 and EP 1871433, can be used as a NO donor. NO releasing polymers can e.g. release NO when contacted with a proton donor. The second reactant can, thus, be a proton donor. In these cases, NO eluted from the polymer is initiated by contact with water or other proton donor and the eluted NO forms a complex with a reducing agent/complexing agent or a complexing agent.

Thus, in one example, the first compartment contains sodium nitrite (e.g. impregnated on a non-woven material) and the second compartment contains a solution of ferrous sulfate (within the container, e.g. within a cavity formed by two wall parts). In other embodiments, the sodium nitrite may be included in the second compartment and the ferrous sulfate may be included in the first compartment.

The first and/or second compartment may include further compounds—in addition to the first reactant and the second reactant—in particular compounds which are directly or indirectly involved in the reaction to generate the active agent. For example, as shown in the above reaction schematics, some reactions for the generation of NO can be supported by further compounds or particular reaction conditions. One or both of the compartments, in particular the second compartment, may, for example include an acid, such as sulfuric acid (H₂SO₄). In other words, the first and/or second compartment may include additional compounds (e.g. additional types of reactants) supporting the reaction to form the active agent.

It should be noted that acid is not required for the production of NO from nitrite and a reducing agent, for example, and large changes in absolute and relative acid concentrations of acid in these reactions do not alter the rates of NO release from a skin dressing. The presence of acid, however, stabilizes reducing agents such as ferrous sulfate, particularly if atmospheric oxygen is present. The reducing agent may be stabilized by the presence of an acid. Acid is in particular not necessary if the device is prepared in a reduced oxygen environment. Acids useful for producing NO and/or stabilizing reducing agents include acetic acid, lactic acid, tartaric acid, ascorbic acid, citric acid salicylic acid, HCl, HBr, H₂SO₄, HNO₃, HClO₄, HI, and combinations thereof.

One or both of the compartments, in particular the second compartment, can additionally include water to improve the distribution of the second reactant within the dressing after rupture of the second compartment.

To enable an efficient reaction of the two reactants after the second compartment is broken, the first and the second compartment are preferably located close to each other within the skin dressing. For example, the second compartment can be located adjacent to the first compartment. The second compartment can be located distally or proximally from the first compartment or within the opening of the first compartment. A location of the second compartment distally from the first compartment is preferred.

The second compartment can be located in a non-centered position relative to a middle axis (M) of the dressing. For example, the second compartment can be located distally or proximally of one part of the first compartment. As the first compartment has an opening in the middle of the dressing, the second compartment does not need to be adjacent to the area of the opening. This does not exclude that the second compartment may overlap with the opening. Nevertheless, it is preferred that 50% or more of the area of the second compartment within the plane that is parallel to the proximal layer and has the greatest extension of the second compartment are directly adjacent to the material of the first compartment, i.e. not overlapping with the opening, e.g. 60% or more, or 70% or more.

The first compartment (its frame structure) may be broader in the area in which the second compartment is adjacent to the first compartment than the remainder of the first compartment. For example, the diameter of an annular structure may be broader.

The skin dressing can be a wound dressing, such as a wound dressing for the treatment of chronic wounds and/or highly exuding wounds. Alternatively, the skin dressing can be a preventative or cosmetic dressing. Such a dressing might be able to improve the health of the skin of the wearer, even in the absence of a distinct wound on the skin. The dressing is adapted for topical delivery of the active agent.

As mentioned before, the skin dressing includes several layers and components—in particular a covering layer, an absorbent layer, a first compartment, a second compartment and a proximal layer. The layers defined herein, such as the covering layer, the absorbent layer and the proximal layer, can be sheet-like layers. The aforementioned layers can have the same size (area measured parallel to the proximal layer). Alternatively, e.g. the covering layer can be larger than the more proximal layers to affix the borders of the covering layer to the skin of the wearer.

The covering layer may be impermeable to the active agent, such as NO-impermeable. In some embodiments, depending on the type of active agent used, the covering layer can be impermeable to gas and fluid. In other embodiments, the covering layer can be impermeable to fluid, but permeable to gas.

Suitable covering layers that have these features are known in the art and can be identified by the skilled person. The covering layer may, for example, consist of or include a material selected from the group consisting of polyurethane and co-polyester or combination thereof, wherein a covering layer including polyurethane is preferred. The polyurethane may be a thermoplastic polyurethane or a non-thermoplastic polyurethane.

The thickness of the covering layer ensures the desired flexibility as well as the desired stability of the covering layer. The layer can have a weight of from 15 to 45 g/m², preferably 20 to 40 g/m², more preferably 25 to 35 g/m².

The covering layer can have an adhesive coating on its proximal side. The adhesive is suitable to connect the covering layer to the more proximal layer(s) and compartment(s). Suitable adhesives for such a coating are known in the art. The adhesive can e.g. be an acrylic adhesive.

The skin dressing furthermore includes an absorbent layer, which is preferably arranged between the covering layer and the first compartment. The absorbent layer includes or consists of one or more absorbent materials. Accordingly, the core is able to take up and retain fluids, such as wound fluids (exudate). The term “wound fluids” or “exudate” refers to fluids which have escaped from a wound, in particular because of inflammatory processes of the blood plasma. The wound fluids or exudates serve to supply the wound bed and the healing processes which are taking place there with a wide array of components, including nutrients for fibroblasts and epithelial cells, growth factors and cytokines. The wound fluids also assist in the cleaning of the wound and the degradation of damaged tissue.

The absorbent material(s) can e.g. include a polymeric foam (such as a hydrophilic foam), a fibrous hydrophilic polymeric material, a hydrogel, a matrix containing hydrocolloids, or combinations thereof. A preferred absorbent material for the absorbent layer is a flexible conformable open-cell foam that is at least slightly hydrophilic. The pore size is uncritical with respect to the other layers; suitable foams have an open cell size of 30-700 micrometers, and preferably a cell size of 50 to 300 micrometers. The absorbent foam may also include a gradient of cell sizes across the thickness of the absorbent core. The open cells permit transport of fluid and cellular debris into and within the foam. The absorption capacity with free swelling of the foams applicable in the disclosure ranges from 5 g/g to 50 g/g if measured according to DIN EN 13726-1 section 3.2, preferably from 10 g/g to 20 g/g.

The absorbent layer may expand about 60-300% its volume when saturated with fluid. It is evident that non-hydrophilic foams may be used as well, where the required absorption is achieved e.g. by capillary action and/or by the incorporation of hydrophilic particles in the foam.

The foam may be made, for example, of polyurethane, cellulose, carboxylated butadienestyrene rubber, polyester foams, hydrophilic epoxy or polyacrylate; wherein polyurethane is preferred.

The thickness of the absorbent layer may range from 0.5 mm to 20 mm, and is preferably from 1 mm to 9 mm, most preferably from 3 mm to 7 mm.

It will be understood that the absorbent material is not limited to being constituted of foam. In an alternative embodiment, the absorbent material may be or can include a hydrophilic porous woven or non-woven fabric material produced by any number of means using known materials available to those skilled in the art. For example, the absorbent material may exist as a bulky, loosely formed web composed of very short cellulosic fibers arranged in a random or non-random array, a pad of cellulose flakes, chitosan flakes, or a polymeric fibril matrix, as well as a mixture thereof. The absorbent layer may also include or consist of superabsorbent particles (SAPs).

The skin dressing further includes a proximal layer which is permeable to the active agent. The proximal layer is the layer which is located most proximal in relation to the skin of the wearer when the dressing is worn correctly. The proximal layer can therefore also be termed wound contact layer or skin contact layer.

The proximal layer may be selectively permeable for the active agent, such as NO. The active agent is allowed to penetrate the proximal layer to the skin while the passage of other, unwanted components or undesired species through the membrane is prevented. The proximal layer can consist of one or more layers, e.g., one or more membranes and/or layers of adhesives or gels.

Accordingly, the proximal layer can include a layer of semi-permeable material. Suitable materials are described, e.g., in EP 3 338 813 B1. The proximal layer may, e.g., include a layer including or consisting of a material selected from the group consisting of polyethylene, polypropylene, viscose, polyethylene terephthalate, and combinations thereof; wherein polyethylene and combinations including polyethylene are particularly preferred. The layer consisting of or including the aforementioned materials can have a thickness of between 20 and 500 μm. Its Vapour Transmission Rate can be between 10 and 120 g/m³*24 h (measured at 37° C., 30% relative humidity).

The proximal layer can include an adhesive on its proximal side. The adhesive is adapted to adhere to the surface of a body part, preferably the skin, of a wearer. It furthermore ensures that the dressing can be removed from the skin substantially without harming the surface of the body part. It has been discovered that a particularly suitable adhesive is an adhesive which includes a silicone. To ensure a firm attachment of the dressing to the skin of the wearer—also during expansion of the absorbent core—the adhesive preferably has a high adhesion to steel value, such as from 0.005 N/cm to 0.8 N/cm, preferably from 0.01 to 0.7 N/cm, such as 0.1 to 0.5 N/cm.

The skin dressing can include one or more channels adapted for the passage of wound exudate. Suitable channels are described in EP 3 338 813 B1 which is incorporated herein in its entirety. The one or more channels extend through the proximal layer and may additionally extend through other layers of the dressing which are located proximally from the absorbent layer. Preferably, the channels extend continuously through all layers which are arranged proximally of the absorbent layer. That way, the channels can efficiently lead wound exudate and other liquid away from the skin or wound and towards the absorbent layer. The one or more channels may be adapted to enable passage of wound exudate from the proximal side of the dressing to the absorbent layer.

The channels can be formed by perforations in the proximal layer and other layers of the dressing which are located proximally from the absorbent layer. To shorten the length of the channels, the perforations in the layers can be arranged on top of each other (forming a “stack of perforations”). This means that the perforations in the layers which together form a channel are adapted and arranged such, that they share a common central axis and have essentially the same diameter.

The term “layer” in this context does not include the first and the second compartment. The perforations in the layers—and therewith the channels—are arranged such that they do not extend through the material of the first compartment or the material of the second compartment. This means that each channel is either running through the opening of the first compartment or located radially outwards from the first compartment. The channels are not arranged directly proximal of the first or the second compartment.

Within the opening of the dressing, the channels can be distributed in a regular manner over the area of the opening.

The perforations can have a circular, triangular, square, rectangular, hexangular or ellipsoid form, wherein a circular or square form is preferred.

In a preferred embodiment the perforations each have an area of between 0.5 mm² and 100 mm². Preference is here given to values of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70,71, 72, 73, 74, 75,76, 77, 78, 79, 80, 81, 82, 83, 84, 85, 86, 87, 88, 89, 90, 91, 92, 93, 94, 95, 96, 97, 98, 99 and/or 100 mm², preferably from 1 mm² to 20 mm², e.g. from 2 mm² to 15 mm². For example, in case the perforations have a circular form (in a plane that is parallel to the proximal layer), the perforations may have a diameter of 0.5 mm or more, 1 mm or more, or 2 mm or more. In other words, the diameter may be from 0.5 mm to 10 mm, from 1 mm to 8 mm, or from 2 mm to 5 mm.

Not all perforations need to be identical in size. The perforations and, thus, channels can have different diameters. The corresponding perforations can, accordingly, each cover areas of different sizes. For example, the channel size can be adapted to the surrounding dressing, e.g. be smaller in the vicinity of the first and second compartment so that they do not interfere with the compartment. But most importantly, the channel diameter/area covered by each perforation can be adapted to the amount of liquid that will have to be taken up the respective channel. Generally, channels with a larger diameter can be present in areas of the dressing in which a lot of wound exudate can be expected and channels with a smaller diameter can be present in areas of the dressing in which less wound exudate can be expected. The dressing may, e.g., have channels with a larger diameter in the middle of the dressing and channels with a smaller diameter in border regions of the dressing.

The perforations can account for, e.g., from 0.1% to 50% of the area of the proximal layer, preferably from 0.5% to 20%, more preferably from 1% to 17,5% and most preferably from 1,5% to 15%, such as from 1,5% to 10%.

The dressing can include 10 or more, 15 or more, preferably 20 or more, channels. In other words, the dressing may include from 10 to 50, from 15 to 45, or from 20 to 40 channels.

The inside of the channels, i.e. the volume in which the liquid is transported through the channels, is preferably sealed off from the first compartment and the second compartment. This ensures that the active agent does not diffuse into the channel interior and that the wound exudate does not leak into the space in the wound dressing that contains the first compartment and the second compartment. The active agent will, thus, leave the dressing through the active-agent permeable proximal layer in the area between the perforations. The area between the perforations within the proximal layer is permeable to the active agent.

To seal the channels off from the space in the wound dressing that contains the first compartment and the second compartment, the layers containing the perforations can be heat sealed or glued together in a rim around each stack of perforations. It is preferred to heat seal the layers containing the perforations together in a rim around each stack of perforations. “Sealing together” means that the layers are fused with each other and/or permanently attached to each other in the respective area. The seal is preferably liquid-tight and can, in addition, be gas-tight.

The layers containing the perforations may be heat sealed or glued together in further parts to optimally distribute and contain the active agent. For example, the layers may be heat sealed or glued together in their margins. This ensures that no active agents escapes to the outside of the dressing. Additionally, the layers may be heat sealed or glued together to form a partial seal around or along the second compartment. Thereby, the active agent can be distributed more evenly over the proximal layer.

In addition to the aforementioned layers, the skin dressing can include a barrier layer that is impermeable to the active agent. The barrier layer is preferably arranged between the absorbent layer and the first compartment. Thereby, an escape of the active agent into the absorbent layer can be prevented and the active agent diffuses solely into the direction of the skin and/or wound of the wearer. The barrier layer can consist of a material that is fluid-impermeable and, depending on the type of active agent used, can also be gas-impermeable. The first compartment and the second compartment are, hence, preferably arranged between the barrier layer and the proximal layer.

The barrier layer can, for example, include or consist of a material selected from the group consisting of EVOH (Ethylene vinyl alcohol), aluminium, aluminium alloys, thicker plastic materials or combinations thereof.

To enable a sufficient uptake of wound exudate, the barrier layer will have perforations that are part of the aforementioned channels. The channels mentioned elsewhere herein, thus, extend through the barrier layer.

It has been outlined before that the area around each channel can be sealed to prevent leakage of the wound exudate and other liquid into the dressing areas around the compartments. For this purpose, the barrier layer and the proximal layer can adhere to each other forming liquid-impermeable barriers around the channels. Thereby, the inside of the channels is separated from the first compartment and the second compartment. Sealing may be performed as described elsewhere herein.

Additional active agents may be included in the first compartment, the second compartment and/or another component of the dressing. Examples of additional active agents include, e.g., antimicrobials and antibiotics. An additional compartment including such additional active agents could be formed within or adjacent to the proximal layer.

The skin dressing can be used in a method of treating a wound, in particular a chronic and/or highly exuding wound. The wound can be a non-healing wound, such as a non-healing ischemic exuding wound, a secondary healing wound, a wound healing by secondary intention, a chronic wound, an infected wound, an exuding wound, an ischemic wound, a neuropathic wound, a burn, a diabetic ulcer, another diabetic wound, a venous and/or arterial leg ulcer, a pressure ulcer, a pressure injury, a decubitus, a mixed ulcer, a trauma wound, a laceration, an abrasion, a cut, a surgical wound, a surgical incision, a skin graft, a donor site or similar or mixtures of the aforementioned types of wounds.

In a second, related aspect a method for producing a skin dressing as described herein includes steps of (i) arranging the proximal layer, the second compartment, the first compartment, the barrier layer, optionally the absorbent layer, and the covering layer on top of each other, thus forming a stack; (ii) forming liquid-impermeable barriers by heating the stack in predetermined areas around the channels and separating the inside of the channels from the first compartment and the second compartment.

The term “stack” describes layers which are lying on top of each other, preferably in a manner that ensures that their perforations are aligned (i.e. each perforation has a common central axis with a perforation in the next layer. Moreover, the layers can have substantially the same outer dimensions so that their edges can also be aligned. The term “aligned” in this context means “on top of each other” (in a direction that is perpendicular to the proximal layer).

It has already been described elsewhere herein that sealing the perforations can create a liquid-impermeable and/or gas-impermeable barrier. The barrier that is created by sealing can be gas tight and/or impermeable to the active agent.

To generate the barrier, the layers are heated in the areas in which the barriers are to be generated. Heating in step (b) can be performed with a stamp-like heating element.

The heating is performed in predetermined areas around the channels. The areas are circumferential rims around the perforations.

### BRIEF DESCRIPTION OF FIGURES

Exemplary embodiments of the invention are shown schematically in the drawings.

FIG. 1 schematically shows a bottom view of one embodiment of a skin dressing;

FIG. 2 schematically shows a bottom view of an alternative embodiment of a skin dressing;

FIG. 3 schematically shows a sectional view of one embodiment of a skin dressing, wherein the dressing includes a barrier layer;

FIG. 4 schematically shows a sectional view of another embodiment of a skin dressing, wherein the dressing includes no barrier layer;

FIG. 5 schematically shows (A) a top view of the secondary compartment and (B) a cross-section of the secondary compartment along the line 2-2 of FIG. 5A; and

FIG. 6 shows an exploded view of the skin dressing shown in FIG. 1.

These and other aspects of the invention will be apparent from and elucidated with reference to the embodiments described hereinafter.

### DESCRIPTION OF EMBODIMENTS

Additional advantages, characteristics, and features of the present invention will become clear from the following detailed description of exemplary embodiments with reference to the attached drawings. However, the invention is not restricted to these exemplary embodiments.

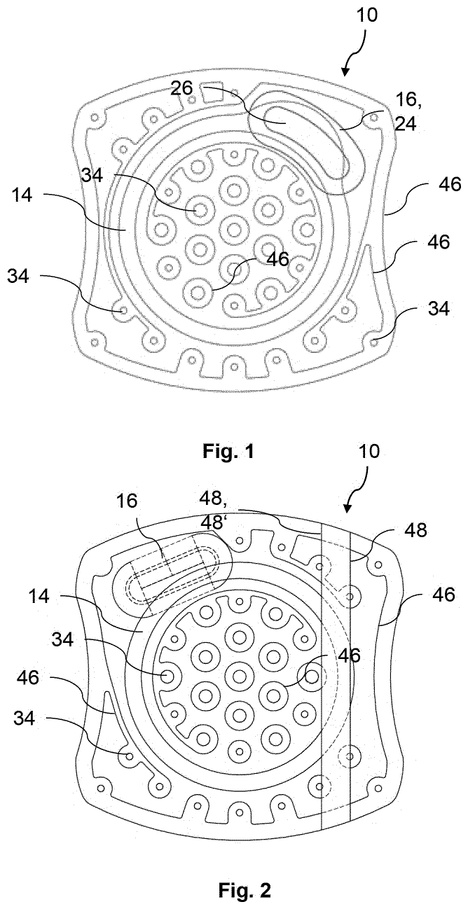

FIG. 1 and FIG. 2 schematically show bottom views of two different embodiments of a skin dressing 10. The skin dressings 10 have a substantially square shape with rounded edges and curved sides. The curvature of the sides is concave on two opposite sides and convex on the other two opposite sides. The skin dressings 10 include several layers and attachment lines 46 at which layers are laminated together, e.g. in the border region of the dressings 10, partially along the first and second compartment and around the channels 34.

In the embodiments shown, the layers that are arranged proximally of the first compartment 14 and the second compartment 16 are transparent or translucent. Therefore, the first compartment 14 and the second compartment 16 are visible through these layers. It can be seen that the skin dressing 10 includes a number of channels 34 which are suitable to facilitate the transport of liquids such as wound exudate through the proximal layer 18 and further layers to the absorbent layer 32. The channels 34 have cylindrical shape, formed by excisions of circular pieces from the layers through which the channels 34 extend and by heat sealing along attachment lines 46.

The channels 34 within the skin dressings 10 have different diameters and cover, thereby, different areas within the layers and have overall different volumes. For example, the channels 34 in the middle of the skin dressing 10 have a larger diameter than the channels in the border regions of the skin dressing 10. Thereby, the wound exudate which is usually present mainly under the middle of the skin dressing 10, can be optimally taken up through these larger channels 34. In the border regions of the skin dressing 10 where less wound exudate is usually present, channels with a smaller diameter are sufficient. Some smaller channels 34 are also present in the middle of the skin dressing 10 to optimally use the space available in the skin dressing 10.

Areas in which at least the layers that are located proximally from the first compartment 14 and the second compartment 16 are laminated together are shown with lines in the FIG. 1. The dressing may include a barrier layer (not shown) that is also laminated to the aforementioned layers along these attachment lines 46. The attachment lines 46 within the skin dressing 10 are formed by heat sealing. Attachment lines 46 are, firstly, formed circularly around the excisions forming some of the channels 34. Additionally, attachment lines 46 are formed in the border region of the dressing to ensure that the active agent does not escape through the sides of the dressing or through the channels 34. Attachment lines 46 are also formed to partially (but not completely) enclose the first compartment 14 and the second compartment 16. Thereby, it is ensured that the first reactant and the second reactant have sufficient opportunity to react with each other to form the active agent before the active agent is released over the entire surface of the proximal layer 18 (not shown).

It can be seen that the first compartment 14 has the shape of a ring, i.e. a circular shape. The ring of the embodiment shown in FIG. 1 has one part that is slightly broader than the rest. This broader part is adjacent to the container that forms the second compartment 16. The part that is adjacent to second compartment 16 includes most of the first reactant in the embodiments shown in FIG. 1 and FIG. 2. The first compartment 14 is formed by a non-woven material that is quickly able to distribute the mixture of first reactant and contents of the second compartment 16 by soaking up the mixture that is generated after breaking of the second compartment 16. Once the mixture is soaked into the entirety of the ring, the active agent is released onto the entirety of the proximal layer-within the exception of areas that are enclosed by attachment lines 46. The active agent can then diffuse through the proximal layer 18 towards the skin and/or wound below the skin dressing 10.

In the embodiment shown in FIG. 2, it can be seen that the skin dressing 10 includes 48, 48′ on its proximal side. The folding lines of one of the release liners 48 are visible in FIG. 2.

The second compartment 16 shown in FIG. 1, FIG. 2 and in more detail in FIG. 5 is a container 24 having an inner cavity 26 in which a solution including the second reactant is present. FIG. 5 schematically shows (A) a top view of the secondary compartment and (B) a cross-section of the secondary compartment along the line 2-2 of FIG. 5A. The walls of the second compartment 16 include a layer including an aluminium composition and are breakable by pushing manually with the hand, in particular e.g. two fingers, of the user. The second compartment has an oval shape (FIG. 2 and FIG. 5) or a kidney shape (FIG. 1) when viewed from the proximal or distal side of the dressing. The second compartment 16 is directly adjacent to the first compartment 14. It overlaps with the first compartment when viewed from proximal or distal side of the dressing.

It can be seen in FIG. 5 that the second compartment 16 consists of a first wall part 28 and a second wall part 30. The first wall part 28 has a flat outer border that can be used for laminating or otherwise attaching the first wall part 28 to the second wall part 30. In its middle, the first wall part includes an indentation (i.e. a depression) that forms an inner cavity 26 once the two wall parts 28, 30 are attached to each other. The inner cavity 26 includes the second reactant.

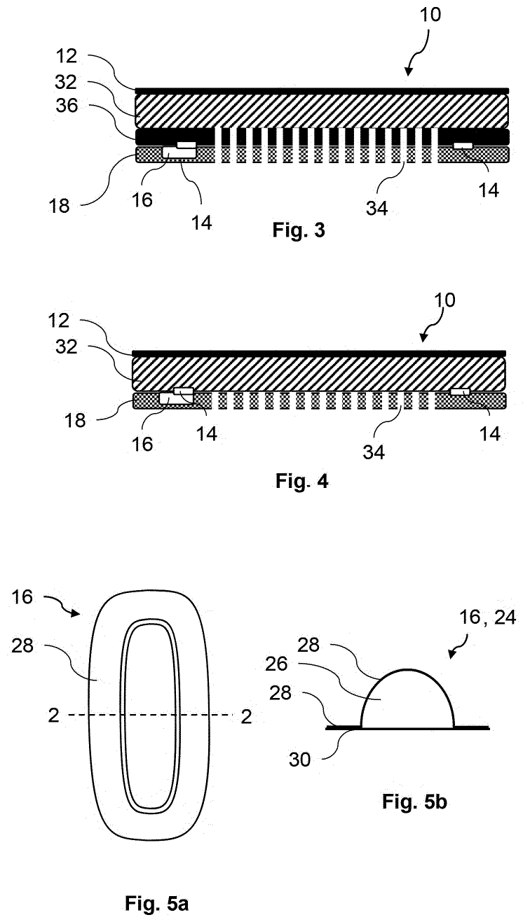

FIG. 3 and FIG. 4 schematically show cross-sectional views of two different embodiments of a skin dressing 10, wherein the skin dressing 10 according to FIG. 3 includes a barrier layer 36 and the skin dressing 10 according to FIG. 4 includes no barrier layer.

The barrier layer 36 of the skin dressing 10 is located distally of the first compartment 14 and the second compartment 16 and impermeable to the active agent (e.g. impermeable to nitric oxide). By blocking the ability of the active agent to diffuse in distal direction, the agent is optimally targeted to the wound or skin surface located proximally of the skin dressing 10. If the active agent is nitric oxide, then the barrier layer 36 is liquid- and gas-tight. The barrier layer can include EVOH and/or polyethylene.

The skin dressings 10 shown in FIG. 3 and FIG. 4 both include-listed from distal to proximal-a covering layer 12, an absorbent layer 32, a first compartment 14, a second compartment 16 and a proximal layer 18. In the dressing 10 of FIG. 3, the barrier layer 36 is located between the absorbent layer 32 and the first compartment 14. The covering layer 12 is a liquid- and gas-impermeable polyurethane layer. It has the same size parallel to the proximal layer 18 as the absorbent layer 32, the barrier layer 36 and the proximal layer 18. The absorbent layer 32 is a hydrophilic polyurethane foam. The foam has good wicking properties and a high absorption capacity.

The proximal layer 18 is the wound- and/or skin contact layer. To ensure that the skin dressing 10 adheres to the skin and/or wound, the proximal layer 18 includes an adhesive or has adhesive properties that allow the skin dressing 10 to adhere. The present proximal layer 18 is, for example, a polyethylene film having a silicone coating or another skin-friendly adhesive on its wound facing side. The proximal layer 18 is—with the exception of course of the channels—preferably impermeable to liquids. It is, however, permeable to the active agent, e.g. for nitric oxide.

It can be seen in FIGS. 3 and 4 that the skin dressings 10 include channels 34 which are suitable to transport exudate away from the proximal side of the dressing towards and into the absorbent layer 32. In the examples shown, the dressings extend through the proximal layer 18 (FIG. 3 and FIG. 4) and the barrier layer 36 (FIG. 3). The first compartment 14 and the second compartment 16 are arranged in a manner that they do not interfere with the channels 34. In other words, when viewed from the proximal side of the dressing, the channels 34 are spaced apart from the (material of the) first compartment 14 and from the second compartment 16. This does not exclude that the channels 34 can extend through the opening 20 within the first compartment. The channels 34 are sealed around each channel, circumferentially around most, so that the wound exudate does not leak into the dressing layers that are located proximally of the absorbent layer 32. Preferably, the active agent does also not leak into the channels 34. In the latter embodiment, the seal is not only liquid-impermeable, but also a gas-impermeable seal.

The first compartment 14 and the second compartment 16 are shown in a schematic side view in FIG. 3 and FIG. 4. The first compartment 14 has a ring-like shape as shown in FIG. 1. In FIGS. 3 and 4 one can see that the second compartment 16 is directly adjacent to (“on top of”) the first compartment 14. Once the second compartment 16 is broken and the fluid which is captured therein released, the fluid will be released onto the first compartment 14. Then, the second reactant included in the second compartment 16 will react with the first reactant included in the first compartment 14 to form the active agent, which is in the present case nitric oxide.

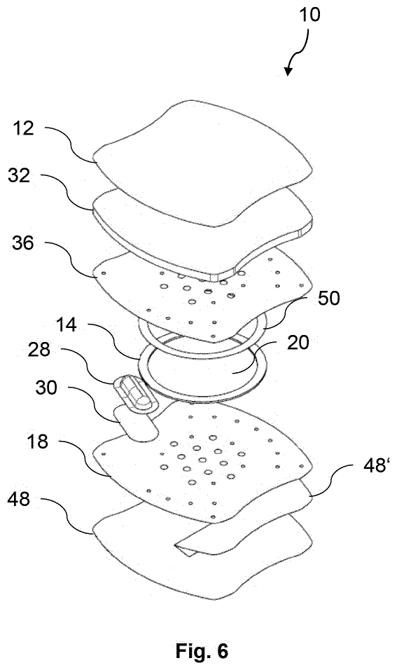

FIG. 6 shows an exploded view of the skin dressing 10 shown in FIG. 2. The skin dressing 10 includes the layers and components that are described above. Moreover, the following more detailed information can be seen in FIG. 6.

The skin dressing 10 includes release liners 48, 48′ which together cover the entire adhesive proximal side of the proximal layer 18. The adhesiveness of the dressing is thereby ensured and the proximal layer 18 protected from dust and other contaminations once the skin dressing 10 is removed from its packaging and from sticking to the packaging. The release liners 48, 48′ can easily be removed by the medical practitioner applying the dressing and are in any case removed before the dressing 10 is attached to the surface of the skin and/or wound.

FIG. 6 further shows that the layers which are located proximally from the absorbent layer 32 have perforations in the same positions relative to the outer borders of the dressing 10. Specifically, the barrier layer 36 and the proximal layer 18 have perforations at the same locations. Additionally, the shape, size and location of the first compartment 14 and the second compartment 16 ensure that these compartments are not at the same positions as the channels relative to the outer borders of the skin dressing 10. Accordingly, these compartments 14, 16 do not interfere with or prevent the formation of channels 34 once the dressing has been assembled. The same applies to optional stabilizing layer 50 which can be a non-woven having the same size and shape as the first compartment 14 when viewed from the proximal layer.

The first wall part 28 and the second wall part 30 which will form the second compartment will be sealed together. Sealing will be performed as heat sealing along the outer border region of both parts. The border region is visible particularly well in the first wall part 28 which has a flat border region and a bulging middle region. After heat sealing of the border regions, the bulging middle region of the first wall part 28 will form the inner cavity 26 of the second compartment 16 together with the second wall part 30. Both wall parts 28, 30 include aluminium and the resulting second compartment 16 is breakable with the force of a thumb or other finger. For assembly, the bulging middle region of the first wall part 28 can be filled to the brim with a solution including the second reactant. Subsequently, the second wall part 30 can be placed onto the first wall part 28 like a lid. Then the border regions of the two wall parts 28, 30 can be heat sealed together to form a breakable capsule. The finalized second compartment 16 can then be assembled with the remaining dressing components, e.g., in the order shown in FIG. 6.

### REFERENCE SIGNS LIST

10 skin dressing
12 covering layer
14 first compartment
16 second compartment
18 proximal layer
20 opening
22 middle region
24 container
26 inner cavity
28 first wall part
30 second wall part
32 absorbent layer
34 channel
36 barrier layer
40 liquid-impermeable barrier
42 inside
44 stack
46 attachment lines
48, 48′ release liner
50 stabilizing layer
M middle axis

1. A skin dressing for topical delivery of an active agent, such as nitric oxide, comprising
a covering layer,
an absorbent layer,
a first compartment comprising a first reactant, wherein the first reactant is a precursor of the active agent,
a second compartment comprising a second reactant, and
a proximal layer permeable to the active agent;
wherein the first compartment has a central opening.

2. The skin dressing according to claim 1, wherein the absorbent layer is arranged between the covering layer and the first compartment.

3. The skin dressing according to claim 1, wherein the first compartment has an annular shape.

4. The skin dressing according to claim 1, wherein the opening has a size of 25% or more of the size of the dressing.

5. The skin dressing according to claim 1, wherein the first compartment comprises a non-woven material.

6. The skin dressing according to claim 1, wherein the second compartment is a container.

7. The skin dressing according to claim 6, wherein the second reactant is comprised in an inner cavity of the container.

8. The skin dressing according to claim 6, wherein the container is adapted to be breakable by external force.

9. The skin dressing according to claim 6, wherein the container comprises a first wall part and a second wall part together enclosing the inner cavity.

10. The skin dressing according to claim 6, wherein the first wall part and/or the second wall part comprise or consist of an aluminium or aluminium alloy.

11. The skin dressing according to claim 1, wherein the second compartment comprises liquid ferrous sulfate solution.

12. The skin dressing according to claim 1, wherein the second compartment is located adjacent to the first compartment.

13. The skin dressing according to claim 1, wherein the second compartment is located in a non-centered position relative to a middle axis of the dressing.

14. The skin dressing according to claim 1, wherein the active agent is nitric oxide.

15. The skin dressing according to claim 1, wherein the dressing comprises one or more channels for the passage of wound exudate.

16. The skin dressing according to claim 15, wherein the one or more channels extend through the proximal layer.

17. The skin dressing according to claim 15, wherein the one or more channels are adapted to enable passage of wound exudate from the proximal side of the dressing to the absorbent layer.

18. The skin dressing according to claim 15, wherein the dressing comprises 15 or more, preferably 20 or more, channels.

19. The skin dressing according to claim 1, comprising a barrier layer impermeable to the active agent.

20. The skin dressing according to claim 19, wherein the barrier layer is arranged between the absorbent layer and the first compartment.

21. The skin dressing according to claim 19, wherein the channels extend through the barrier layer.

22. The skin dressing according to claim 19, wherein the first compartment and the second compartment are arranged between the liquid permeable barrier layer and the proximal layer.

23. The skin dressing according to claim 19, wherein the barrier layer and the proximal layer adhere to each other forming liquid-impermeable barriers around the channels separating the inside of the channels from the first compartment and the second compartment.

24. A method for producing a skin dressing according to claim 1, comprising steps of
(i) arranging the proximal layer, the second compartment, the first compartment, the barrier layer, optionally the absorbent layer, and the covering layer on top of each other, thus forming a stack;
(ii) forming liquid-impermeable barriers by heating the stack in predetermined areas around the channels and separating the inside of the channels from the first compartment and the second compartment.

25. The method according to claim 24, wherein heating in step is performed with a stamp-like heating element.
